(19) United States
(12) Patent Application Publication
(10) Pub. No.: US 2016/0375057 A1
(43) Pub. Date: Dec. 29, 2016

(54) THERAPEUTIC COMPOSITIONS AND METHODS INVOLVING CELL-PERMEABLE IRON

(71) Applicant: REGENTS OF THE UNIVERSITY OF MINNESOTA, MINNEAPOLIS, MN (US)

(72) Inventors: Sundaram Ramakrishnan, Minneapolis, MN (US); Devika Kir, New Haven, CT (US); Sabita Roy, Minneapolis, MN (US)

(21) Appl. No.: 15/189,886

(22) Filed: Jun. 22, 2016

Related U.S. Application Data
(60) Provisional application No. 62/183,801, filed on Jun. 24, 2015.

(51) Int. Cl. A61K 33/26; A61K 31/282; A61K 31/704; A61K 31/664

(52) U.S. Cl. CPC A61K 33/26; A61K 31/664; A61K 31/282; A61K 31/704; A61N 2005/1098

(57) ABSTRACT

This disclosure describes, in one aspect, therapeutic methods that involve administering cell-permeable iron to a subject. In some cases, the cell-permeable iron may be co-administered to the subject with a chemotherapeutic agent or radiation treatment, each in an amount that, in combination with the other, is effective to ameliorate at least one symptom or clinical sign of the tumor. In other cases, the cell-permeable iron may be administered in an amount effective to inhibit angiogenesis.

### CROSS-REFERENCE TO RELATED APPLICATION

This application claims priority to U.S. Provisional Patent Application No. 62/183,801, filed Jun. 24, 2015, which is incorporated herein by reference.

### GOVERNMENT FUNDING

This invention was made with government support under RO1 DA031201-01 awarded by the National Institutes of Health. The government has certain rights in the invention.

### SUMMARY

This disclosure describes, in one aspect, a method for treating a subject having a tumor. Generally, the method includes co-administering to the subject cell-permeable iron and a chemotherapeutic agent or radiation treatment, each in an amount that, in combination with the other, is effective to ameliorate at least one symptom or clinical sign of the tumor.

In another aspect, this disclosure describes a method of inhibiting angiogenesis in a subject. Generally, the method includes administering to the subject a composition that includes cell-permeable iron in an amount effective to inhibit angiogenesis.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Data show that OC494 cells (mucinous ovarian cancer cells), which are highly resistant the current methods of chemotherapy, can be sensitized to mafosfamide by cell-permeable iron (e.g., ferric ammonium citrate, FAC). Mafosfamide, alone, did not kill the cells even at 100 μM. In the presence of FAC, significant cell death was noticed even at 10 μM mafosfamide. FAC in combination with 100 μM mafosfamide showed more than 75% inhibition of OC494 cells.

FIG. 2. Ovarian cancer cells were treated with varying concentrations of carboplatin, one of the standard chemotherapeutic drugs used for ovarian cancer. More than 50% of patients treated with carboplatin develop resistance. Cell-permeable iron (ferric ammonium citrate, FAC) increased sensitivity to carboplatin by three-fold. Cells are killed at lower concentration of carboplatin in the presence of cell-permeable iron.

FIG. 3. Synergy between carboplatin and cell-permeable iron. Cytotoxicity was determined by CCK8 assay after 24 hours. Dotted line shows theoretical prediction of additive effect. The hypobolic curve confirms synergy between the two reagents. (Chou et al., 2006, Pharmacol Rev 58:621-628).

FIG. 4. Synergy between an alkylating agent, mafosfamide and ferric ammonium citrate was determined in xCELLigence System (Roche Diagnostics, Indianapolis, Ind.) which measures cell proliferation in real time. CI, combination index. CI<1.0 indicates synergy. Compusyn program (ComboSyn, Inc., Paramus, N.J.) was used to calculate CI and generate isobologram. MA148 ovarian cancer cells were used in this study.

FIG. 5. Synergy between various concentrations of ferric ammonium citrate and various concentrations of doxorubicin. Dotted line shows theoretical prediction of additive effect. The hypobolic curve confirms synergy between the two reagents. (Chou et al., 2006, Pharmacol Rev 58:621-628).

FIG. 6. Cell-permeable iron, FAC, inhibits cancer-stem-cell-rich spheroid formation. Ovarian cancer cells were grown in conditions to favor cancer-stem-cell-rich spheroid formation. FAC treatment inhibited the size and viability of the cancer-stem-cell-rich spheroids. Propidium iodide uptake (fluorescence) shows dead cells. Control spheroids are viable (negative for propidium iodide staining).

FIG. 7. Cell-permeable iron, FAC, inhibits angiogenesis. Inhibition of VEGF-induced human umbilical vein endothelial cell proliferation is inhibited by FAC treatment.

FIG. 8. Cell-permeable iron (ferric ammonium citrate, FAC) treatment inhibits VEGF-receptor signaling. VEGF induced phosphorylation of VEGFR-2 and downstream signaling through Erk phosphorylation are inhibited by FAC. Representative western blot show inhibition of VEGFR-2 signaling by FAC.

FIG. 9. Cell-permeable iron (FAC) inhibits human umbilical vein endothelial cell migration. Angiogenesis involves cell proliferation and migration. FAC treatment was found to inhibit VEGF-induced endothelial cell migration in scratch wound assays. Data are from three independent experiments and error bars show standards deviation.

FIG. 10. Cell-permeable iron, FAC, inhibits endothelial cell migration as determined by electrical impedance measurements in real time.

FIG. 11. Systemic administration of cell-permeable iron (FAC) inhibits tumor angiogenesis in mice. Tumor cells were injected into groups of mice subcutaneously in MATRIGEL plugs. Intraperitoneal treatment with FAC decreased tumor blood vessels. Data show blood vessel staining (arrows, CD31-positive endothelial cells). Blue staining shows tumor cell nuclei. Histograms show mean levels of blood vessel length and branch points.

FIG. 12. Cell-permeable iron (FAC) sensitizes human breast cancer cells to radiation therapy. Triple negative breast cancer cell line, MDA-MB-231-luc, was exposed to different doses of ionizing radiation (X-Ray) in the presence and absence of 100 μM FAC. Sets of cultures seeded at the same cell density were kept as a control for radiation-induced cell death. Radiation alone showed marginal effect on cell viability (nuclei are shown as white dots). FAC alone without any exposure to radiation showed significant decrease in cell viability. FAC treated cultures when exposed to two Gy and four Gy radiations almost completely killed all the treated cells. These data show that FAC treatment sensitizes breast cancer cells to radiation induced cell death.

FIG. 13. FAC inhibits the clonogenic growth of lymphoma cells. Burkitt's B-lymphoma cells were treated with FAC at different concentrations. Treated cells grown for 14 days at different cell density (2 cells/well-50 cells/well). Left panel shows phase contrast images of control and FAC treated cultures. Colony formation, which reflects on cancer initiating stem cells, was completely inhibited by FAC treatment. Right panel shows quantification of FAC effects on long-term growth of lymphoma cells.

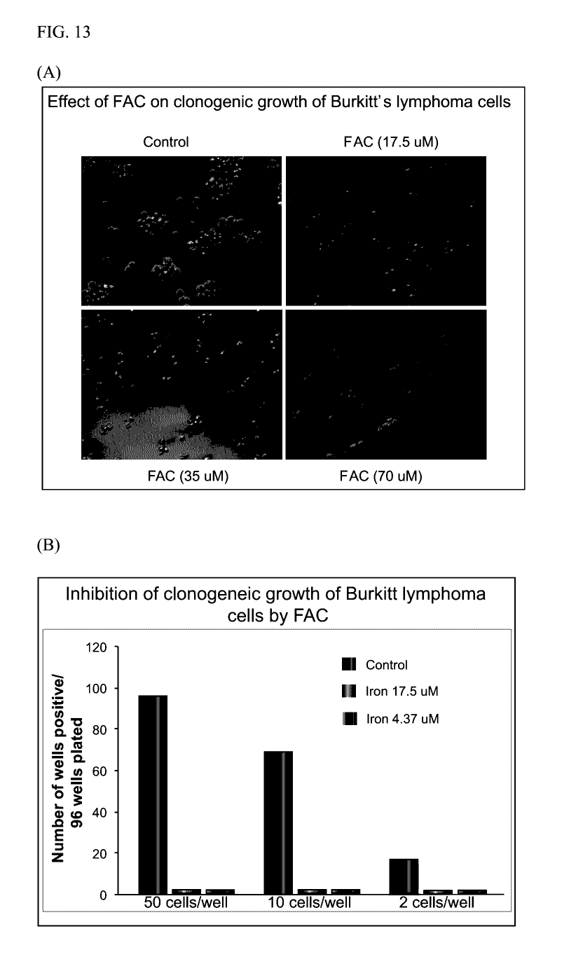

FIG. 14. Ferric ammonium citrate (FAC) is more potent than ferric citrate (FC) at inhibiting lymphoma cell growth in vitro. Burkitt's lymphoma cells were treated with either ferric ammonium citrate (FAC) or ferric citrate (FC). Both forms of cell-permeable iron were effective inhibiting lymphoma cell growth, but FAC was more effective than FC.

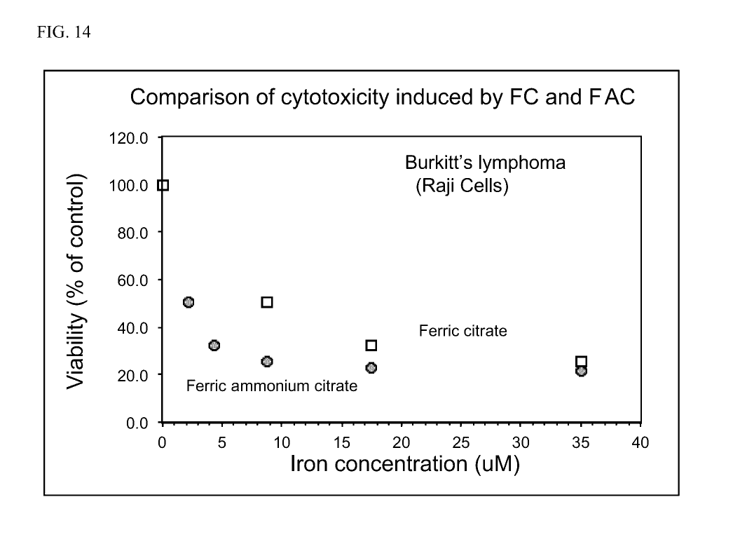

FIG. 15. FAC potentiates the effect of topoisomerase inhibitor, etoposide. Ovarian cancer cells were treated either with etoposide alone or in combination with FAC. These studies showed FAC potentiated the effect of etoposide.

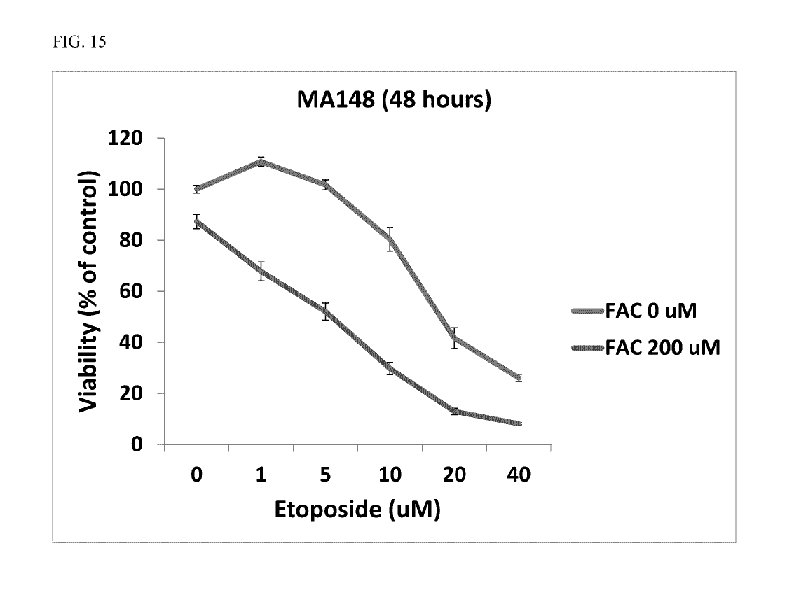

FIG. 16. FAC potentiated the therapeutic effect of carboplatin against human ovarian cancer. Athymic mice were transplanted with MA148 human ovarian cancer cells. After two weeks to allow tumors to establish, mice were divided into four groups. Group 1 was treated with saline solution and served as a control. Group 2 mice were treated with 6.8 mg/kg/day FAC intraperitoneally for three weeks. Group 3 mice were treated with carboplatin (6.8 mg/kg, twice a week for three weeks). Group 4 mice were treated with a combination of FAC and carboplatin at similar dosages. These studies demonstrated that FAC treatment improved the anti-tumor effects of carboplatin in vivo.

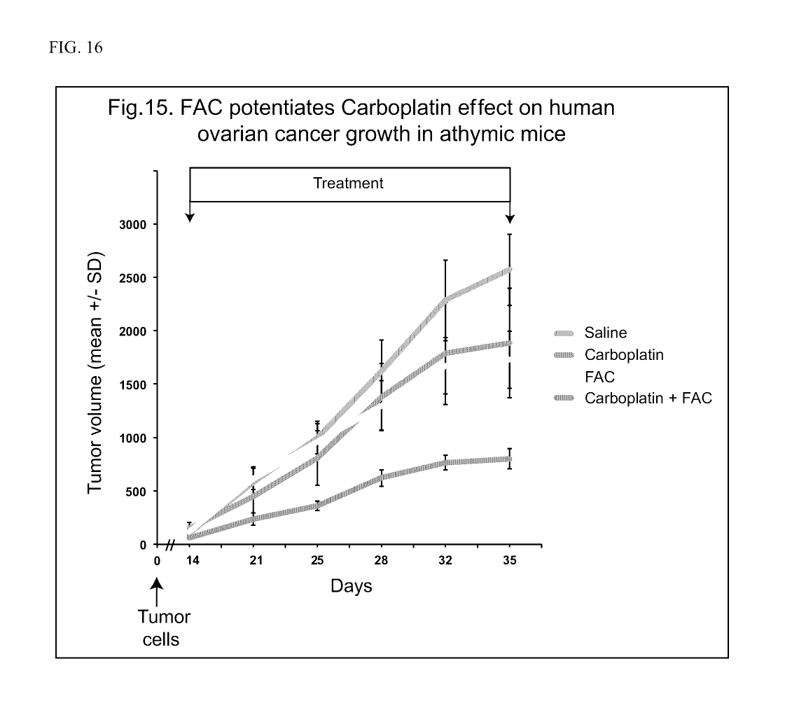

### DETAILED DESCRIPTION OF ILLUSTRATIVE EMBODIMENTS

This disclosure describes, generally, methods and compositions that involve the use of cell-permeable iron to limit cell growth. In one aspect, the methods and compositions can involve using cell-permeable iron to sensitize tumor cells to chemotherapeutic drugs so that the combination produces a synergistic effect. In another aspect, the methods and compositions can involve using cell-permeable iron to inhibit angiogenesis and, as a result, can be used therapeutically to treat a condition that is promoted by angiogenesis. An additional use of cell-permeable iron is that it can be used as a radiation sensitizer to improve the effect of radiation therapy. Cell-permeable iron can be used to treat tumors in human and animals either alone or in combination with chemotherapy, anti-angiogenic therapy, and/or radiation therapy.

Molecular iron is involved in various cellular functions and metabolic processes. For example, iron-sulfur complexes are central to the mitochondrial respiratory complex, oxygen transport, and DNA polymerases. While a trace amount of iron is required to maintain cellular homeostasis, excessive iron is toxic to cells. Excessive iron can generate reactive hydroxyl and oxygen radicals by Fenton/Haber Weiss reaction. Iron levels are therefore tightly regulated in the cells by controlling processes involved in iron uptake, storage, and export. Total iron uptake in humans is modulated by the liver-gut axis. Iron is transported into cells by receptor-mediated endocytosis of transferrin. Downregulation of endocytosis during hypoxia leads to lower iron levels in cells with concomitant stabilization of hypoxia inducible factors, HIF-1α and HIF-2α.

Cancer cell proliferation and migration are inhibited by cell-permeable iron (e.g., ferric ammonium citrate) in a concentration dependent manner. Administering a cell-permeable form of iron can increase the cytotoxicity of, for example, a cancer chemotherapeutic agent.

Thus, in one aspect, this disclosure describes a method of providing treatment to a subject that includes co-administering to a subject a cell-permeable form of iron and a chemotherapeutic agent or radiation. As used herein, “co-administer” and variations thereof refer to two or more components of a combination administered so that the therapeutic or prophylactic effects of the combination can be greater than the therapeutic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another.

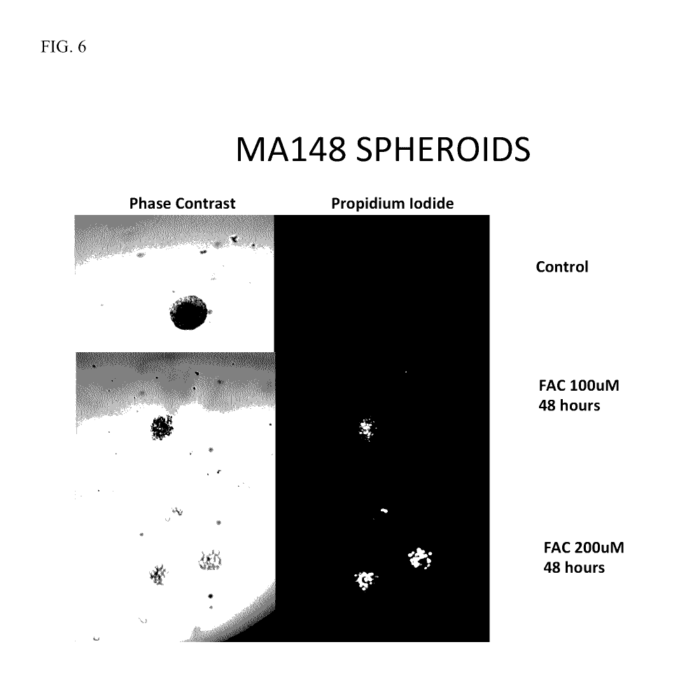

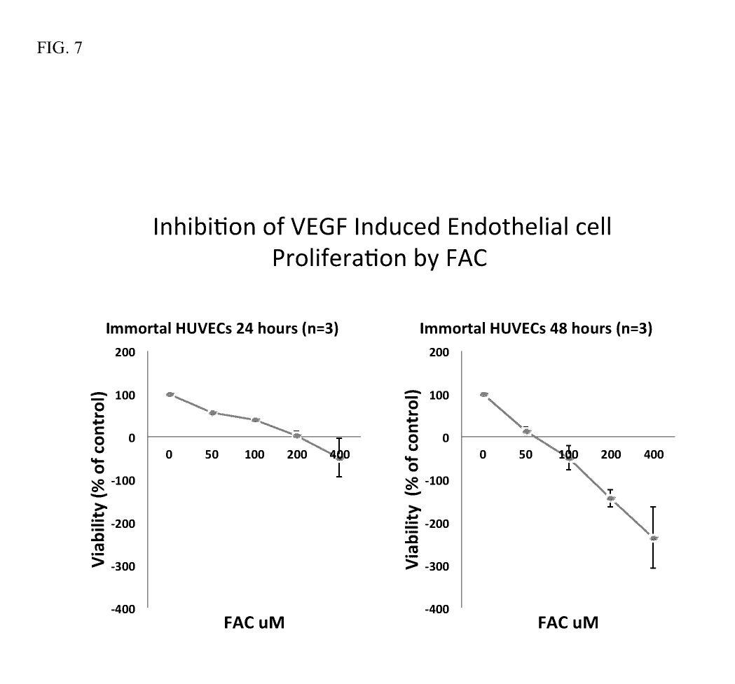

In another aspect, this disclosure describes methods of inhibiting angiogenesis in a subject. Generally, the methods involve co-administering cell-permeable iron and an anti-angiogenic chemotherapeutic agent. The model cell-permeable iron, ferric ammonium citrate (FAC), markedly inhibited endothelial cell migration (FIG. 6). Furthermore, systemic injection of FAC blocked tumor angiogenesis in MATRIGEL plug assays (Corning, Inc., Corning, N.Y.). CD31 positive vessel density was significantly reduced by FAC when compared to mice receiving saline. (FIG. 7).

Inhibiting angiogenesis can, therefore, inhibit tumorigenesis, tumor growth, and/or metastasis. Angiogenesis is, however, involved in conditions other than solid tumor forms of cancer. Inhibiting angiogenesis can therefore be useful in treating any condition that involves, at least in part, an elevated level of angiogenesis, for example, macular degeneration, diabetes-induced retinopathy, rheumatoid arthritis, and/or psoriasis.

Thus, this disclosure generally describes methods that involve co-administering to a subject a cell-permeable form of iron and a chemotherapeutic agent. In some cases, the chemotherapeutic agent may be designed to treat a neoplastic condition such as, for example, a tumor. The methods may be used to treat benign or malignant tumors. Moreover, the methods may be used to treat solid tumors or liquid tumors (e.g., leukemias or lymphomas). In other cases, the chemotherapeutic agent may be designed to treat an angiogenic condition.

In some cases, co-administering a cell-permeable form of iron and a chemotherapeutic agent can result in a synergistic effect. As used herein, a “synergistic” effect refers to activity that is greater than the expected additive effect of the co-administered components if each of the components had been administered alone. One method of detecting a synergistic effect involves computing a combination index (CI) using, for example, COMPUSYN software (ComboSyn, Inc., Paramus, N.J.). Generating a synergistic effect can allow effective chemotherapy using a smaller amount of chemotherapeutic agent, thereby reducing cost, improving patient compliance, reduce the likelihood of tumor cells developing resistance to the chemotherapeutic agent, and/or decreasing the likelihood and/or severity of side effects associated with larger doses of the chemotherapeutic agent. In some cases, co-treatment with radiation can result in synergistic effect with cell-permeable iron. Cell-permeable iron can be used to pretreat and sensitize tumor cells of human and animals to radiation therapy.

Conventional cancer treatments (e.g., chemotherapy and radiation) can fail to completely cure the cancer. Recurrence of cancer is initiated by cancer stem cells that survive anti-cancer therapy. Cancer stem cells typically reside in hypoxic areas of the tumors. Cell-permeable iron inhibits cancer-stem-cell-rich spheroids. Thus, cell-permeable iron can be used to reduce the likelihood of tumor recurrence when used either alone or in combination with chemotherapy or radiation therapy.

Cancer chemotherapy can lead to multiple drug resistance. One resistance mechanism involves inhibition of drug uptake. Iron nanoparticles have been used in, for example, imaging and treatments for cancer. To the extent that the iron nanoparticles are internalized into cells, the internalization is dependent upon endocytosis. Endocytosis is inhibited at hypoxic regions of the tumor. Thus, hypoxic regions of a tumor can decrease endocytosis and provide a sanctuary for cancer stem cells against therapies that involve endocytosis.

In contrast, cell-permeable iron can bypass the uptake inhibition mechanism of drug resistance. The uptake of cell-permeable forms of iron is independent of endocytosis. Thus, co-administering a cell-permeable form of iron with a chemotherapeutic agent can sensitize, for example, cancer stem cells to the chemotherapy even in a hypoxic region of a tumor where endocytosis is diminished.

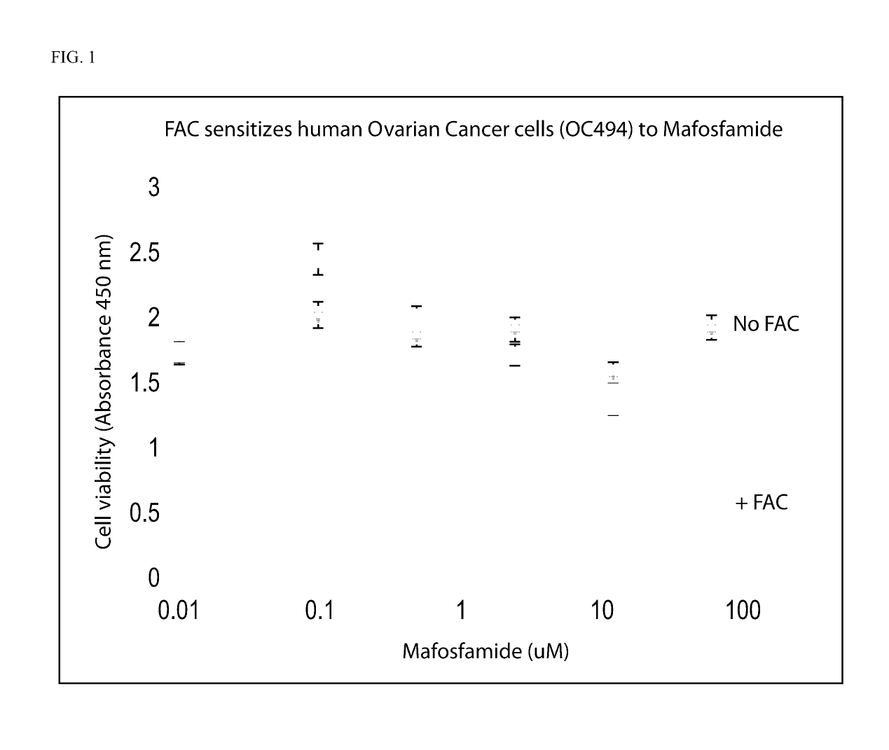

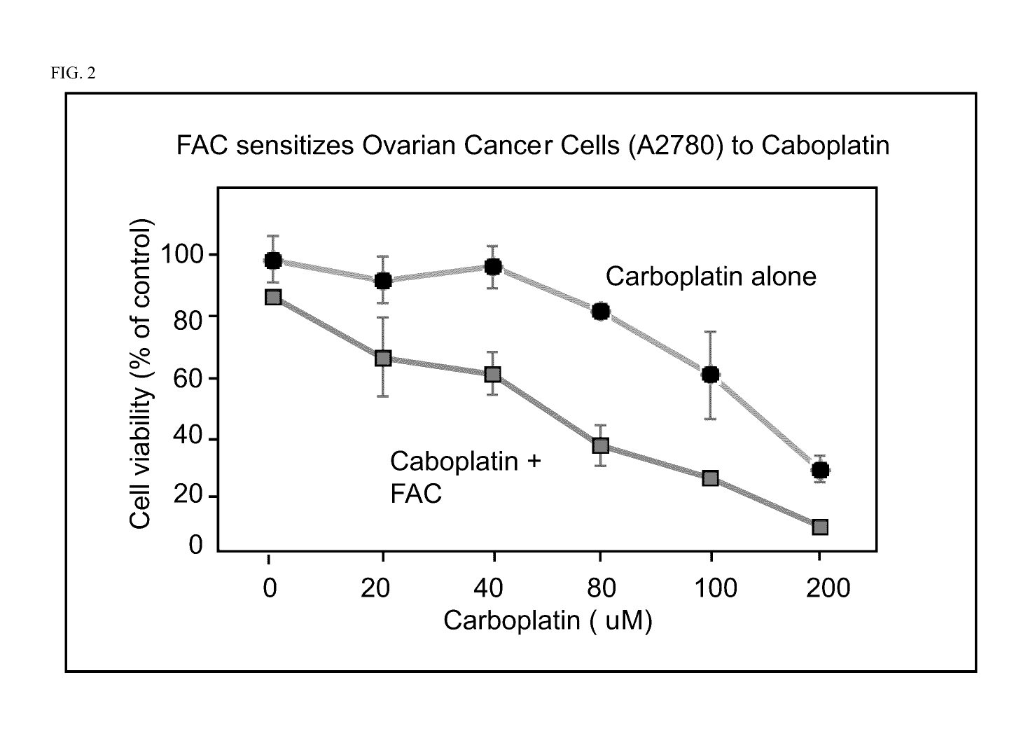

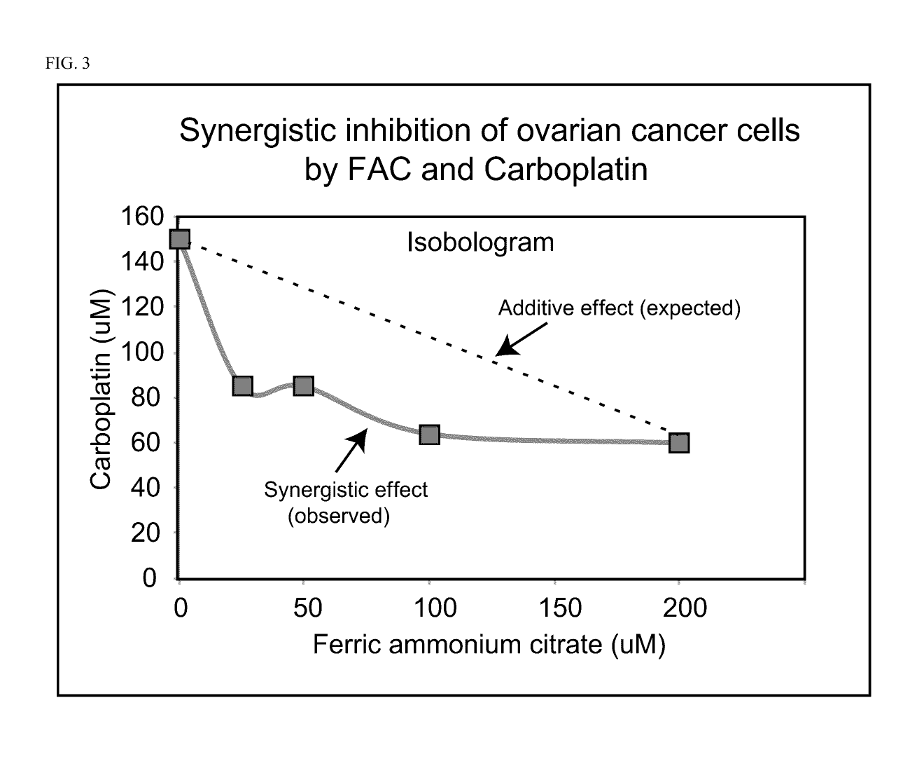

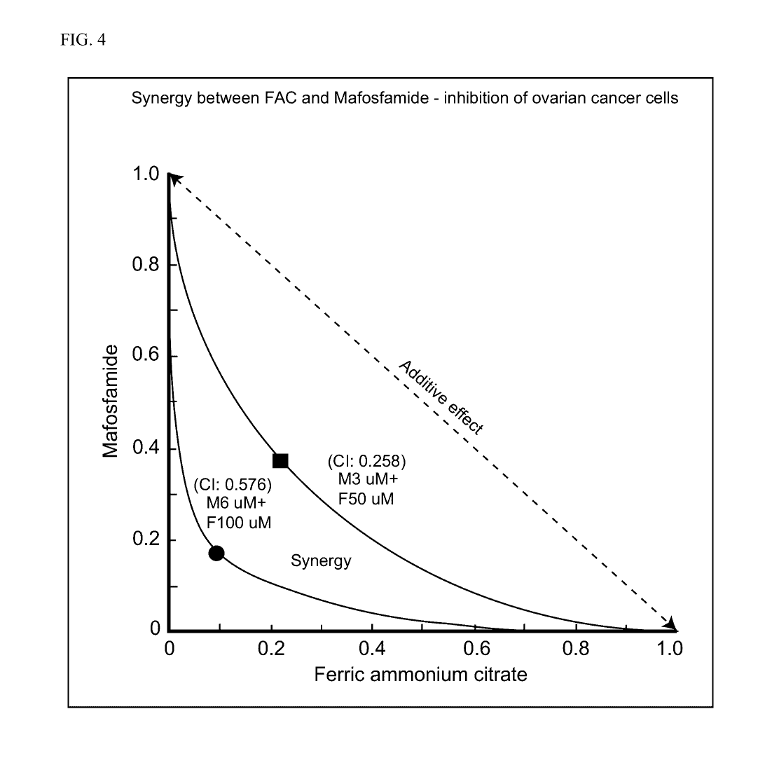

FIG. 1 and FIG. 2 show data that demonstrate that cell-permeable iron sensitizes cancer cells to model chemotherapeutic agents carboplatin and mafosfamide. Tables 1-4, FIG. 3, and FIG. 4 show data that establishes that the effect of co-administering cell-permeable iron and the same model chemotherapeutic agents produces a synergistic effect. Isobolograms, such as those shown in FIG. 3 and FIG. 4, are conventionally used to show synergy between two reagents. In these experiments, varying concentrations of the two reagents are added to tumor cells in an array format. Cytotoxicity was determined by CCK8 assay after 24 hours. The dotted lines show theoretical prediction of additive effect. The data show a hypobolic curve, confirming synergy between the two reagents. An antagonistic effect (competition) would result in hyperbolic (bell-shaped) curve above the dotted line (additive effect).

TABLE 1
Combination Index − Mafosfamide + Ferric ammonium citrate
CI Data for Non-Constant Combo: MAFFAC (MAF + FAC)

| Dose MAF | Dose FAC | Effect | CI* |
|---|---|---|---|
| 25.0 | 100.0 | 0.24288 | 0.90196 |
| 50.0 | 100.0 | 0.24359 | 0.97391 |
| 75.0 | 100.0 | 0.28253 | 0.78143 |
| 100.0 | 100.0 | 0.29392 | 0.77283 |
| 200.0 | 100.0 | 0.66880 | 0.09603 |
| 25.0 | 200.0 | 0.35541 | 0.77717 |
| 50.0 | 200.0 | 0.36087 | 0.78413 |
| 75.0 | 200.0 | 0.49929 | 0.35376 |
| 100.0 | 200.0 | 0.61254 | 0.18678 |
| 200.0 | 200.0 | 0.87322 | 0.02473 |

*CI—Combination index <1.0 indicates synergy between the drug combination.

TABLE 2
Dose Reduction Index (DRI*) − Mafosfamide + Ferric ammonium citrate
DRI Data for Non-Constant Combo: MAFFAC (MAF + FAC)

| Fa | Dose MAF | Dose FAC | DRI *MAF | DRI* FAC |
|---|---|---|---|---|
| 0.24288 | 322.441 | 121.297 | 12.8976 | 1.21297 |
| 0.24359 | 324.287 | 121.991 | 6.48574 | 1.21991 |
| 0.28253 | 436.171 | 164.074 | 5.81561 | 1.64074 |
| 0.29392 | 473.376 | 178.068 | 4.73376 | 1.78068 |
| 0.66880 | 4852.00 | 1824.65 | 24.2600 | 18.2465 |
| 0.35541 | 716.325 | 269.444 | 28.6530 | 1.34722 |
| 0.36087 | 741.848 | 279.043 | 14.8370 | 1.39522 |
| 0.49929 | 1715.18 | 645.092 | 22.8690 | 3.22546 |
| 0.61254 | 3382.59 | 1272.12 | 33.8259 | 6.36059 |
| 0.87322 | 29593.1 | 11126.4 | 147.965 | 55.6319 |

*Dose Reduction Index (DRI) >1.0 indicates potential benefit of reducing dose and decreased toxicity by the combination of drugs.

TABLE 3
Combination Index − Doxorubicin + Ferric ammonium citrate
CI Data for Non-Constant Combo: DOXF48 (DOX + FAC)

| Dose DOX | Dose FAC | Effect | CI* |
|---|---|---|---|
| 200.0 | 200.0 | 0.88386 | 0.07988 |
| 500.0 | 200.0 | 0.95196 | 0.03581 |

*CI—Combination index <1.0 indicates synergy between the drug combination.

TABLE 4
Dose Reduction Index (DRI*) − Doxorubicin + Ferric ammonium citrate
DRI Data for Non-Constant Combo: DOXF48 (DOX + FAC)

| Fa | Dose DOX | Dose FAC | DRI* DOX | DRI* FAC |
|---|---|---|---|---|
| 0.88386 | 47050.0 | 2644.47 | 235.250 | 13.2223 |
| 0.95196 | 178095. | 6060.28 | 356.189 | 30.3014 |

*Dose Reduction Index (DRI) >1.0 indicates potential benefit of reducing dose and decreased toxicity by the combination of drugs.

In some embodiments, a treatment method can include co-administering a cell-permeable form of iron and a chemotherapeutic agent. As used herein, the term “treat” and variations thereof refer to reducing, limiting progression, ameliorating, or resolving, to any extent, the symptoms or signs related to a condition. As used herein, “ameliorate” refers to any reduction in the extent, severity, frequency, and/or likelihood of a symptom or clinical sign characteristic of a particular condition. “Symptom” refers to any subjective evidence of disease or of a patient's condition. “Sign” or “clinical sign” refers to an objective physical finding relating to a particular condition capable of being found by one other than the patient.

The cell-permeable iron may be any form of iron that may be taken up by the cell in a manner that does not require endocytosis. Exemplary cell-permeable forms of iron include, for example, ferric ammonium citrate (FAC), ferric citrate (FC), iron complexed with tricarboxylic acids (e.g., citric acid, citric acid ammonium salt, 1,2,3-propanetricarboxylic acid), ferrous chloride, ferric chloride, ferrous sulfate, ferric sulfate, ferric salts of ethylenediaminetetraacetic acid (EDTA), iron lignin sulfonate, iron metaphosphate, ammonium iron(II) sulfate hexahydrate, dichlorotetrakis(pyridine)iron, iron(II) bromide (FeBr₂), iron(III) bromide (FeBr₃), iron(II) chloride (FeCl₂), iron(III) citrate, iron(II) fluoride (FeF₂), iron(III) fluoride (FeF₃), iron(II) iodide anhydrousIron(II) molybdate (FeMoO₄), iron(III) nitrate nonahydrate (Fe(NO₃)₃.9H₂O), iron(II) oxalate dihydrate (FeC₂O₄.2H₂O), iron(II) perchlorate hydrate (Fe(ClO₄)₂.xH₂O), iron(III) phosphate tetrahydrate (FePO₄.4H₂O), iron(III) pyrophosphate (Fe₄(P₂O₇)₃), iron(II) sulfate hydrate (FeSO₄.xH₂O), and iron(II) tetrafluoroborate hexahydrate (Fe(BF₄)₂.6H₂O).

The amount of cell-permeable iron administered can vary depending on various factors including, but not limited to, the specific cell-permeable iron used, the weight, physical condition, and/or age of the subject, and/or the route of administration. Thus, the absolute weight of cell-permeable iron included in a given unit dosage form can vary widely, and depends upon factors such as the species, age, weight and physical condition of the subject, and/or the method of administration. Accordingly, it is not practical to set forth generally the amount that constitutes an amount of cell-permeable iron effective for all possible applications. Those of ordinary skill in the art, however, can readily determine the appropriate amount with due consideration of such factors.

In certain embodiments, the cell-permeable iron can be administered at a minimum dose of at least 0.1 g/day/65 kg such as, for example, 0.2 g/day/65 kg, at least 0.5 g/day/65 kg, at least 1.0 g/day/65 kg, at least 2.0 g/day/65 kg, at least 3.0 g/day/65 kg, at least 4.0 g/day/65 kg, at least 5.0 g/day/65 kg, or at least 6.0 g/day/65 kg. In certain embodiments, the cell-permeable iron can be administered at a maximum dose of no more than 20.0 g/day/65 kg such as, for example, no more than 10 g/day/65 kg, no more than 9.0 g/day/65 kg, no more than 8.0 g/day/65 kg, n more than 7.0 g/day/65 kg, no more than 5.0 g/day/65 kg, no more than 4.0 g/day/65 kg, no more than 3.0 g/day/65 kg, n more than 2.0 g/day/65 kg, or no more than 1.0 g/day/65 kg. In some embodiments, the cell-permeable iron can be administered at a dose expressed as a range have endpoints defined by any minimum dose listed above and any maximum dose listed above that is greater than the minimum dose. Thus, for example, in one particular embodiment, the cell-permeable iron can be administered at a dose of from 0.2 g/day/65 kg to 10 g/day/65 kg such as, for example, a dose of from 2.0 g/day/65 kg to 6.0 g/day/65 kg.

The chemotherapeutic agents can be any suitable chemotherapeutic agent. Exemplary chemotherapeutic agents include, for example, an alkylating agent, an antimetabolite, an anthracycline, an anti-tumor antibiotic, a topoisomerase inhibitor, a mitotic inhibitor, a kinase inhibitor, an anti-angiogenic therapeutic, an immunotherapeutic, a cancer-stem-cell-directed drug, an anti-inflammatory drug, and/or a drugs directed to modulate tumor microenvironment.

Exemplary alkylating agents include, for example, a nitrogen mustard such as, for example, mechlorethamine (nitrogen mustard), chlorambucil, cyclophosphamide, ifosfamide, and melphalan; a nitrosourea such as, for example, streptozocin, carmustine (BCNU), and lomustine; an alkyl sulfonate such as, for example, busulfan; a triazine such as, for example, dacarbazine (DTIC) and temozolomide; or an ethylenimine such as, for example, thiotepa and altretamine (hexamethylmelamine). The platinum-based drugs (i.e., platins) such as, for example cisplatin, carboplatin, and oxalaplatin, are sometimes grouped with alkylating agents because they kill cells in a similar way. Thus, as used herein, the term alkylating agent includes platinum-based drugs.

Antimetabolites include compounds that interfere with DNA and/or RNA by substituting for nucleotide bases during the production of DNA or RNA. Exemplary antimetabolites include, for example, 5-fluorouracil (5-FU), 6-mercaptopurine (6-MP), capecitabine, cytarabine, floxuridine, fludarabine, gemcitabine, hydroxyurea, methotrexate, and pemetrexed.

Anthracyclines are anti-tumor antibiotics that interfere with enzymes involved in DNA replication. These drugs work in all phases of the cell cycle. They are widely used for a variety of cancers. Exemplary anthracyclines include, for example, daunorubicin, doxorubicin, epirubicin, and idarubicin. One concern associated with the use of anthracyclines is that they can permanently damage the heart if given in high doses. For this reason, lifetime dose limits are often placed on these drugs. Accordingly, co-administering an anthracycline with cell-permeable iron can allow one to use lower doses of the drug and thereby increase the time over which the drug may be used before reaching the lifetime dosing limit.

Exemplary alternative anti-tumor antibiotics include, for example, actinomycin-D, bleomycin, mitomycin-C, and mitoxantrone.

Topoisomerase inhibitors interfere with the enzymatic activity of topoisomerases, which help separate the strands of DNA so they can be copied during the S phase. Topoisomerase inhibitors are used to treat certain leukemias, as well as lung, ovarian, gastrointestinal, and other cancers. Topoisomerase inhibitors are generally grouped according to whether they inhibit topoisomerase I or topoisomerase II. Exemplary topoisomerase I inhibitors include, for example, topotecan and irinotecan (CPT-11). Exemplary topoisomerase II inhibitors include, for example, toposide (VP-16), teniposide, etoposide, and mitoxantrone.

Mitotic inhibitors include compounds that stop mitosis in the M phase of the cell cycle, but can damage cells in any phase by inhibiting cells from making proteins needed for cell reproduction. Mitotic inhibitors includes plant alkaloids. Exemplary mitotic inhibitors include, for example, taxanes such as, for example, paclitaxel and docetaxel; epothilones such as, for example, ixabepilone; vinca alkaloids such as, for example, vinblastine, vincristine, and vinorelbine; and estramustine.

Exemplary kinase inhibitors include, for example, imatinib, gefitinib, sunitinib, and bortezomib.

Exemplary anti-angiogenic therapeutics include, for example, endostatin, angiostatin, canstatin, antibody that targets VEGF or VEGF receptor, VEGF-Trap, and/or inhibitors of VEGF, chemokines, HGF, PDGF, or FGF signaling.

Exemplary therapeutics include, for example, an immune checkpoint inhibitor, a CAR T cell, a cytokine, or a chemokine.

The amount of chemotherapeutic agent administered can vary depending on various factors including, but not limited to, the specific chemotherapeutic agent, the weight, physical condition, and/or age of the subject, and/or the route of administration. Thus, the absolute weight of chemotherapeutic agent included in a given unit dosage form can vary widely, and depends upon factors such as the species, age, weight and physical condition of the subject, and/or the method of administration. Accordingly, it is not practical to set forth generally the amount that constitutes an amount of chemotherapeutic agent effective for all possible applications. Those of ordinary skill in the art, however, can readily determine the appropriate amount with due consideration of such factors.

For example, certain chemotherapeutic agents may be administered at the same dose and frequency for which the drug has received regulatory approval. In other cases, the chemotherapeutic agent may be administered at the same dose and frequency at which the drug is being evaluated in clinical or preclinical studies. One can alter the dosages and/or frequency as needed to achieve a desired level of therapeutic effect when co-administered with the cell-permeable iron. Thus, it is possible that a desired therapeutic effect may be achieved using less chemotherapeutic agent than is conventionally used. Moreover, one can use standard/known dosing regimens and/or customize dosing as needed.

The cell-permeable iron and the chemotherapeutic agent may be formulated with a pharmaceutically acceptable carrier. The formulation can include the cell-permeable iron and the chemotherapeutic agent. Alternatively, the cell-permeable iron and chemotherapeutic agent may be formulated separate from one another. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredients—i.e., the cell-permeable iron and the chemotherapeutic agent—its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the active agents without causing any undesirable biological effects or interacting in a deleterious manner with any of the other components of the pharmaceutical composition in which it is contained.

The active agents may therefore be formulated into a pharmaceutical composition. As used herein, “active agent” refers to a cell-permeable form of iron, a chemotherapeutic agent, or a combination thereof. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the active agents may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

As noted above, the cell-permeable iron and the chemotherapeutic agent can be administered together or separate and, therefore, may be formulated together or separately. Also, the cell-permeable iron and the chemotherapeutic agent, when administered separately, may be provided by different routes of administration.

The subject treated as described above can be any suitable animal subject including, for example, a human, a companion animal (e.g., a dog or a cat), a livestock animal (e.g., a horse, cow, goat, pig, or sheep). Thus, the methods described herein can be practiced in the context of human and/or veterinary medicine.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Human ovarian cancer cells established from a mucinous histological subtype (OC494 cells, Dr. O. Martinez-Maza, University of California, Los Angeles) were seeded into 96-well plate at a density of 10,000 cells (180 μl) per well in RPMI-1640 medium supplemented with 10% (v/v) Newborn bovine serum (NBS), 100 U/ml penicillin and 100 μg/ml of streptomycin (complete medium). Cells were allowed to attach and grow for 24-hours. Mafosfamide was weighed and made into a stock solution of 38 mM and filter sterilized using a 0.22 μm membrane. Mafosfamide was then serially diluted in complete medium and added to five wells for each concentration. 100 μM (final concentration/well) ferric ammonium citrate (FAC), made in complete medium, was added to the wells. After incubation at 37° C. for 48 hours, cell viability was determined by Cell Counting Kit-8 (CCK-8, Dojindo Molecular Technologies, Inc., Rockville, Md.) viability assay kit. Three hours after the addition of CCK-8 (20 μl/well), absorbance was determined at 450 nm. Data in FIG. 1 show changes in absorbance (viability) of mafosfamide in the presence and absence of FAC-treated ovarian cancer cells. Error bars represent Standard Deviation (SD). Cultures treated with mafosfamide alone did not show any change in viability over a concentration range of 0.01 μM to 60 μM. However, in the presence of FAC, mafosfamide significantly inhibited the viability of ovarian cancer cells in the concentration range of 2.4 μM to 60 μM. At 60 μM concentration of Mafosfamide and 100 μM FAC, complete inhibition of ovarian cancer cells was observed.

### Example 2

FIG. 2 shows FAC treatment sensitizes human ovarian cancer cells (A2780 cells, Dr. Thomas Hamilton, Fox Chase Cancer Center, Philadelphia, Pa.) to carboplatin-mediated cytotoxicity. A2780 cells were cultured in 96-well plates. Culture conditions used were similar to Example 1. Sets of five wells were treated either with carboplatin alone (20 μM -200 μM) or in combination with FAC (100 μM). Cell viability was determined using CCK-8 kit (Dojindo Molecular Technologies, Inc., Rockville, Md.) after 48 hour-treatment. Absorbance at 450 nm of control cultures was considered 100 per cent viability. Error bars represent standard deviation. FAC potentiated Carboplatin-induced cytotoxicity of ovarian cancer cells.

### Example 3

Synergistic inhibition of ovarian cancer cells by FAC and carboplatin. A2780 ovarian cancer cells were seeded into 96-well plates. Carboplatin was added to sets of cultures at concentrations between 60 μM and 150 μM. Another series of cultures were treated similarly, but in the presence of 25 μM, 50 μM, 100 μM, or 200 μM ferric ammonium citrate. Cell viability was determined by CCK-8 kit after 48-hour treatment with the drugs. The concentration of carboplatin required to inhibit 50% cell viability (compared to control cultures, IC₅₀) were calculated. IC₅₀ values are shown as individual data points. IC₅₀ of carboplatin alone showed a value of 150 μM. IC₅₀ of FAC alone was 200 μM in this experiment. The dotted line shows predicted additive effect between carboplatin and FAC. Values bellow the dotted line indicate synergy between the two compounds to inhibit ovarian cancer cells.

### Example 4

FAC synergizes with mafosfamide in inhibiting ovarian cancer cells. Human ovarian cancer cells, MA148 (Dr. S. Ramakrishnan, University of Minnesota-Twin Cities, Minneapolis, Minn.), were plated in 96 well plates (5000 cells/well). Cells were allowed to attach for 24 hours after which they were pretreated with FAC 200 μM for 24 hours. Subsequently, they were treated with different concentrations of mafosfamide in the presence or absence of FAC 100 μM. After 24 hours, CCK-8 (10 μl) was added to each well and one hour later, absorbance at 450 nm was measured. Viability was calculated as percentage of the cells grown in complete medium (FIG. 4). Two different combinations of FAC and mafosfamide were used in this study. Synergy between mafosfamide and ferric ammonium citrate was determined using Compusyn program (ComboSyn, Inc., Paramus, N.J.). Isobologram was generated using Compusyn program (ComboSyn, Inc., Paramus, N.J.). Dotted line is the predicted additive effect between two compounds. Values below the dotted line indicate synergy.

### Example 5

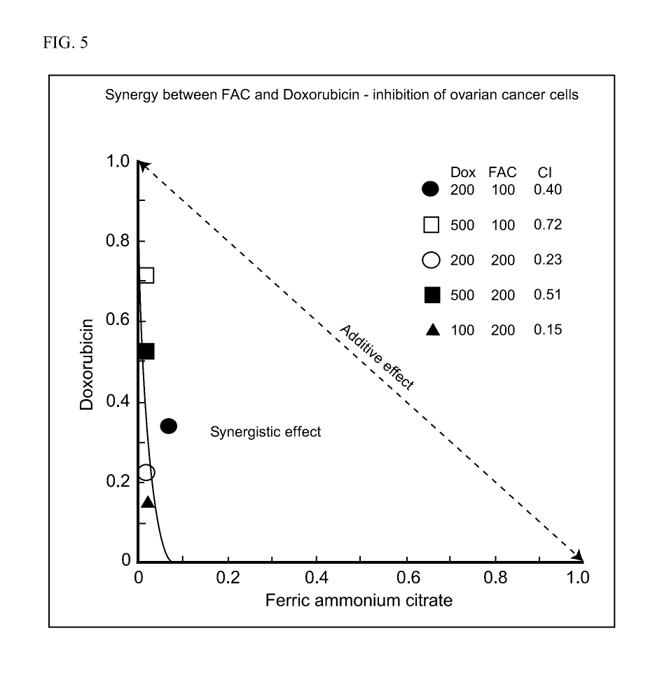

FAC synergizes with doxorubicin in inhibiting ovarian cancer cells. Human ovarian cancer cells, MA148 (Dr. S. Ramakrishnan, University of Minnesota-Twin Cities, Minneapolis, Minn.), were plated in 96 well plates (5000 cells/well). Cells were allowed to attach for 24 hours after which they were pretreated with FAC 200 μM for 24 hours. Subsequently, they were treated with different concentrations of Doxorubicin in the presence or absence of FAC 200 uM. After 24 hours, CCK-8 (10 μl) was added to each well and one hour later, absorbance at 450 nm was measured. Viability was calculated as percentage of the cells grown in complete medium (FIG. 5). Five different combinations of FAC and Doxorubicin were used in this study. Synergy between doxorubicin and ferric ammonium citrate was determined using Compusyn program (ComboSyn, Inc., Paramus, N.J.). Combination index (CI) and Isobologram was generated using Compusyn program (ComboSyn, Inc., Paramus, N.J.). Dotted line is the predicted additive effect between two compounds. Values below the dotted line indicate synergy.

### Example 6

FAC treatment inhibits cancer-stem-cell-rich spheroid formation. Ovarian cancer cells, MA148 (Dr. S. Ramakrishnan, University of Minnesota-Twin Cities, Minneapolis, Minn.), were grown in non-adherent tissue culture dishes for one week. Stem-cell-rich spheroids were then separated by bovine serum albumin (1.0% in sterile Hanks Balanced Salt Solution) gradient at unit gravity. Purified spheroids were then treated with FAC and then cultured for 48 hours. Data in FIG. 6 show viability of cancer-stem-cell-rich spheroids. Control, untreated spheroids were large and viable (negative for propidium iodide staining), whereas FAC-treated cultures showed smaller spheroids and are positive for propidium iodide staining indicating cell death. These studies show that FAC inhibits cancer-stem-cell-rich spheroid formation.

### Example 7

### FAC Treatment Inhibits Angiogenesis

### Animals, Cell Lines and Reagents

Human umbilical vein endothelial cells (HUVECs; Neuromics, Edina, Minn.) and HUVECs immortalized with h-TERT (ATCC CRL-4053, ATCC, Manassas, Va.) were cultured in endothelial cell growth media EGM-2 (Lonza, Basel, Switzerland). Lewis Lung Carcinoma (LLC) cells (ATCC CRL-1642, ATCC, Manassas, Va.) were grown in RPMI 1640 medium supplemented with 10% FBS, 1% glutamine, 1% penicillin-streptomycin and 1% sodium pyruvate. Female C57BL/6 wild-type (WT) mice were used as recipients (bred at the University of Minnesota specific pathogen free environment animal facility). All animals were sex and aged matched (6-8 weeks). All experimental protocols were approved by the University of Minnesota Institutional Animal Care and Use Committee. VEGF-A₁₆₅ was purchased from R & D Systems (Minneapolis, Minn.). GELTREX Low-growth factor basement membrane matrix (Thermo Fisher Scientific, Inc., Waltham Mass.) was used for in vitro tube formation assay. MATRIGEL basement membrane matrix (Corning, Inc., Corning, N.Y.) was used for LLC MATRIGEL plug assays.

Antibodies used were: anti-ERK1/2 (Cell Signaling Technology, Inc., Danvers, Mass.), anti-phosphor-ERK1/2 (Cell Signaling Technology, Inc., Danvers, Mass.), anti-VEGFR-2 (FLK-1) (Santa Cruz Biotechnology, Inc., Dallas, Tex.), anti-pY1175 VEGFR-2 (Cell Signaling Technology, Inc., Danvers, Mass.), anti-GAPDH (EMD Millipore Corp., Merck KGaA, Darmstadt, Germany), PE-conjugated anti-CD31 (BD Biosciences, San Jose, Calif.), FITC-conjugated anti-alpha SMA (Sigma-Aldrich, St. Louis, Mo.). Appropriate secondary antibodies tagged to horseradish peroxidase (Vector Laboratories, Inc., Burlingame, Calif.) were used. Fluorescent probes Hoescht-33342, 4′,6-diamidino-2-phenylindole (DAPI), and propidium iodide (PI) were from Molecular Probes (Thermo Fisher Scientific, Inc., Waltham Mass.).

Ferric ammonium citrate (FAC) and deferoxamine mesylate (DFX) were purchased from Sigma-Aldrich (St. Louis, Mo.).

### FAC Inhibits VEGF-A-Induced Endothelial Cell Proliferation

HUVEC were plated in 0.2% gelatin coated 96-well plates and allowed to attach for 24 hours. Subsequently they were starved in EBM-2 with 2% serum overnight. After 16 hours, cells were treated with different concentrations of FAC in EBM-2 with 2% serum and 100 ng/ml VEGF-A for 24 hours or 48 hours. Appropriate negative (no VEGF-A) and positive controls (100 ng/ml VEGF) were used. All treatments were done in triplicates. Cell viability was determined by CCK-8 assay (Dojindo Molecular Technologies, Inc., Rockville, Md.). VEGF-A induced effect was calculated by subtracting negative control values from all experimental groups. Proliferation was then calculated as percentage of the positive control. Data in FIG. 7 show concentration dependent inhibition of VEGF-induced cell proliferation of immortalized human umbilical vein endothelial cells. 24 hours of treatment with 200 μM FAC completely inhibited VEGF-A-induced endothelial cell proliferation. At 48 hours of treatment, 50 μM FAC showed complete inhibition of VEGF-A-induced cell proliferation. These studies show FAC inhibits angiogenesis.

### FAC Treatment Inhibits VEGF-A₁₆₅ Induced Signaling in Human Endothelial Cells

HUVECs were plated in 0.2% gelatin-coated 60 mm dishes. Once 75% confluent, they were growth factor starved in EBM-2 with 2% serum overnight with/without 200 μM FAC. After 16 hours of starvation, they were stimulated with 100 ng/ml VEGF-A with/ without 200 μM FAC and lysates were collected at 0 minutes, 10 minutes, 30 minutes, or 60 minutes. Cells were lysed with 100 μL RIPA buffer (Alfa Aesar, Ward Hill, Mass.) containing protease and phosphatase inhibitor cocktail (Thermo Fisher Scientific, Inc., Waltham, Mass.). Protein concentration was measured using BCA Protein Assay Kit (Pierce, Thermo Fisher Scientific, Inc., Waltham, Mass.) and 30 μg of protein was loaded. The samples were resolved in 7.5% (ERK1/2,P-ERK1/2) and 15% (VEGFR2) SDS-PAGE gels. The resolved proteins were transferred to a 0.45 μm nitrocellulose membrane (Bio-Rad Laboratories, Inc., Hercules, Calif.) and blocked with 5% non-fat milk or 5% BSA in TBST (for phosphor-proteins) for one hour at room temperature.

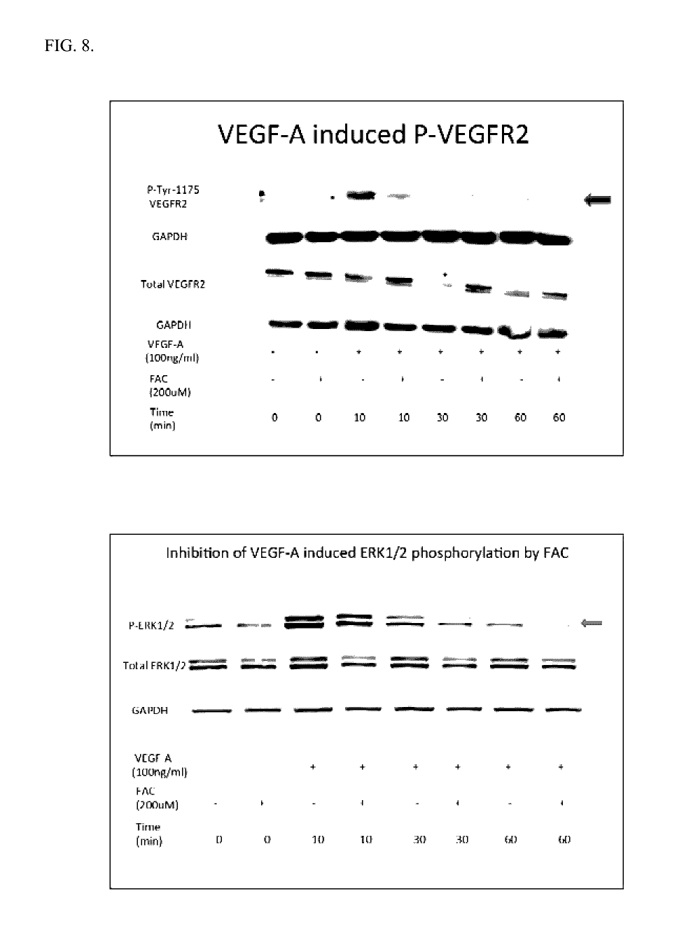

The membrane was incubated with primary antibody ERK (1:1000), P-ERK (1:1000), PY1175 (1:1000), VEGFR-2 (1:1000), GAPDH (1:1000), or VEGFR-2 (1:250) overnight at 4° C. Membranes were then treated with anti-mouse or rabbit IgG secondary antibodies (1:1000) conjugated with horseradish peroxidase. Immunoblots were detected with CLARITY Western ECL Substrate (Bio-Rad Laboratories, Inc., Hercules, Calif.) using enhanced chemiluminescence technique. For ERK1/2 and P-ERK1/2, IR fluorescence was detected using an ODYSSEY imaging system (LI-COR, Inc., Lincoln, Nebr.). Quantification of bands was done using ImageJ (Schneider et al., 2012, Nature methods 9(7):671-675). Data in FIG. 8 show representative images of VEGF receptor signaling. VEGF-A binding to VEGF receptor-2 (VEGFR-2) induces receptor tyrosine phosphorylation and stimulates downstream phosphorylation of Erk signaling. Phosphorylated VEGFR-2 and phosphorylated Erk levels are decreased by FAC treatment. Arrows show the phosphorylated protein bands from representative western blots.

### FAC Treatment Inhibits VEGF-A₁₆₅ Induced Migration (Scratch Wound Assay) of Human Endothelial Cells

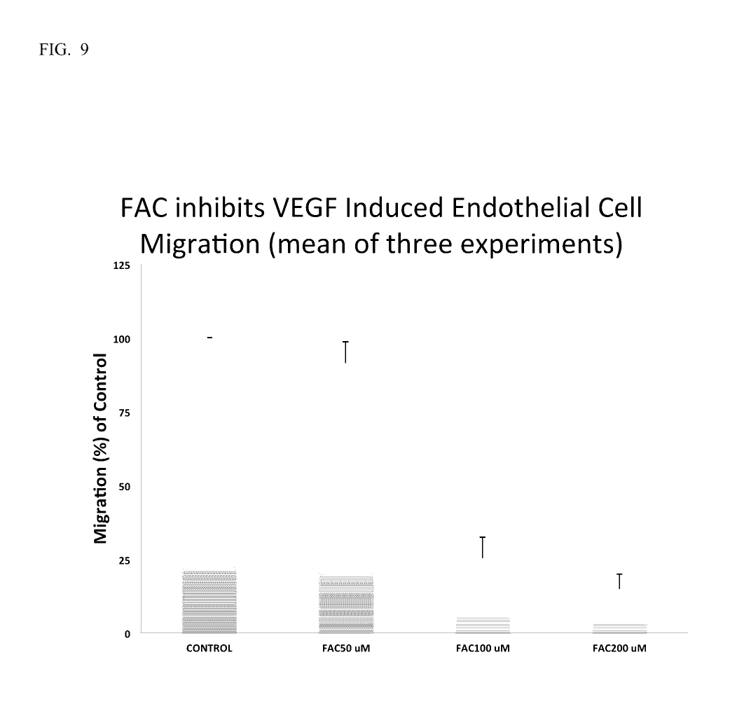

HUVEC (50,000 cells) were plated in 0.2% gelatin-coated 24 well plates and allowed to attach for 24 hours. Subsequently, they were growth factor starved in EBM-2 with 2% serum overnight. After 16 hours of starvation, a scratch was made with a 1000 μL pipette tip and 0-hour photos were taken at 4× magnification with the Nikon AZ 100M Macro scope. The cells were treated with different concentrations of FAC in EBM-2 with 2% serum and 100 ng/ml VEGF-A. Appropriate negative (no VEGF-A) and positive controls (100 ng/ml VEGF) were used. All treatments were done in triplicates. At 24 hours, the experiment was arrested and cells were fixed with 4% paraformaldehyde and stained with Hoescht-33342. Images of the scratch wound were taken at 4× magnification with the Nikon AZ 100M Fluorescence Macro scope after 24 hours. Closure of the scratch wound area was determined by ImageJ software (Schneider et al., 2012, Nature methods 9(7):671-675). Migration was calculated as a percent of the initial wound area. Data in FIG. 9 show FAC treatment inhibits endothelial cell migration induced by VEGF treatment in scratch wound assays. Mean values from three independent experiments are shown. Error bars indicate standard deviation.

### FAC Inhibits Endothelial Cell Migration in Complete Medium.

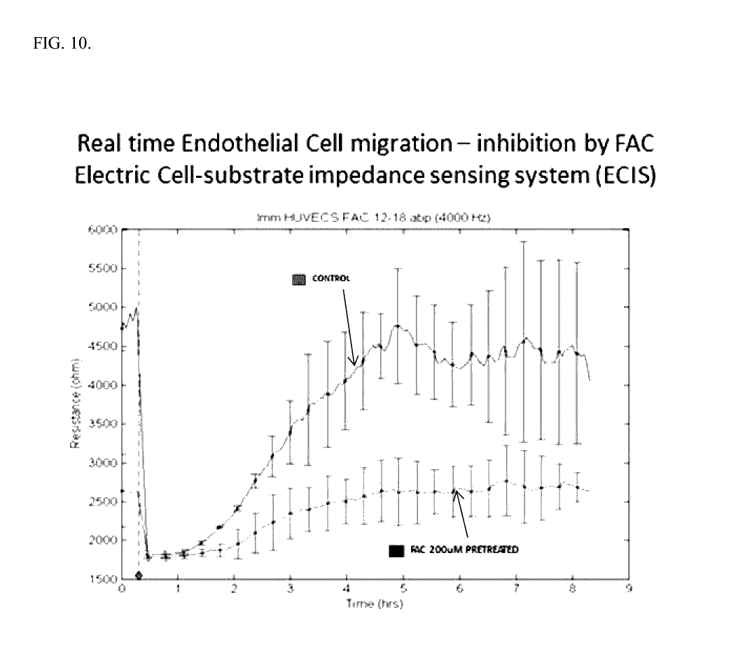

HUVECs were plated in 60 mm plates and allowed to grow and attach for 24 hours. Subsequently, one plate was treated with FAC 200 μM for 24 hours. Control and treated cells were trypsinized and 50,000 live cells were plated in 8 well (8W10E) electric cell substrate impedance sensing plate (ECIS, Applied BioPhysics, Inc., Troy, N.Y.). Cell attachment and spreading was confirmed by measuring electrical impedance. After reaching stable values of impedance in ohms, a clear wound of 250 micrometer was generated by applying elevated electrical pulse to the defined electrode. Wounded area is clear of cells and confirmed by a decrease in impedance. Cell movement into the wounded areas was recorded for eight hours at 15 minute intervals. Cell migration into the wounded area increases impedance and was continually recorded over a period of eight hours (FIG. 10). Error bars represent standard deviation (SD).

### FAC Treatment Inhibits Tumor Angiogenesis

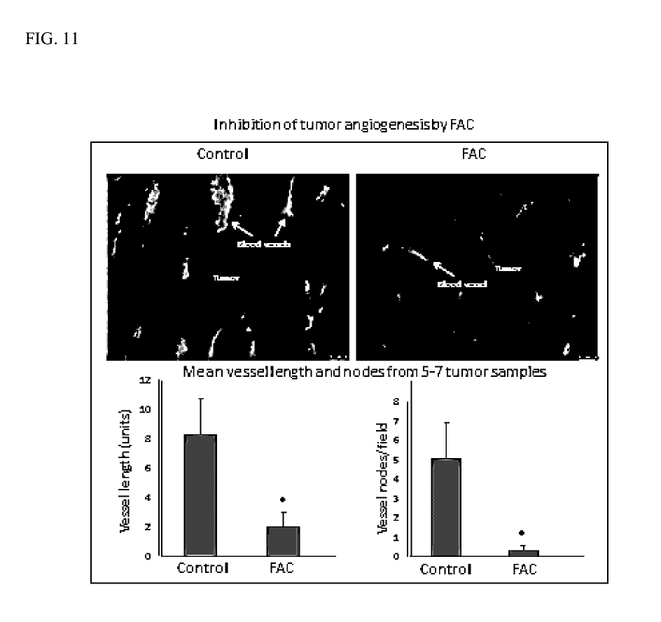

One million LLC cells (in PBS) were mixed with MATRIGEL (Corning, Inc., Corning, N.Y.) in a 3:1 ratio and injected subcutaneously into the dorsal flanks (bilateral sites) of 6-8 week old female C57BL/6 wild-type mice. Experimental MATRIGEL plugs contained 200 μM FAC. Four days later, mice were treated with either saline (negative control) or 20 mg/kg FAC (in PBS) on alternate days for two weeks. After two weeks, mice were sacrificed, tumor plugs removed and photographed, a part of the MATRIGEL plugs was snap-frozen, and another part of the MATRIGEL plugs was fixed in 10% neutral formalin. Frozen tumor plugs (n=5 for saline and n=7 for FAC) were sectioned (15 μm), fixed with acetone at −20° C. for 10 minutes, rehydrated, and blocked in 1% BSA. Slides were then stained with PE-conjugated anti-mouse CD31 (1:100) overnight at 4° C., washed three times with PBS, stained with FITC-conjugated anti-alpha-SMA (1:500) for one hour at room temperature and counterstained with DAPI. Two sections per tumor plug were stained. Random areas (5-20) were imaged at 10× magnification using a Nikon AZ 100M Fluorescence Macro scope and at 20× magnification using a Leica DM5500 B microscope. CD31 vessel length and branching points (nodes) were quantified by morphometric analyses of fluorescent digital images using Reindeer Games plug-in functions for Adobe Photoshop, as previously described (Wild et al., 2000, Microvac Res 59(3):368-376). CD31 positive vessels co-staining with alpha- SMA were quantified similarly to quantitate mature vessels. Data in FIG. 11 show representative images of tumor sections stained with rat anti-mouse CD31-phycoerythrin conjugate. Red staining shows CD31-positive blood vessels. Blue staining shows tumor cell nuclei. FAC treatment of mice significantly inhibited number of blood vessels in tumor sections. Total vessel length and vessel branches are inhibited by FAC treatment.

### Example 8

### FAC Treatment Potentiates Radiation-Induced Cell Death of Human Breast Cancer Cells.

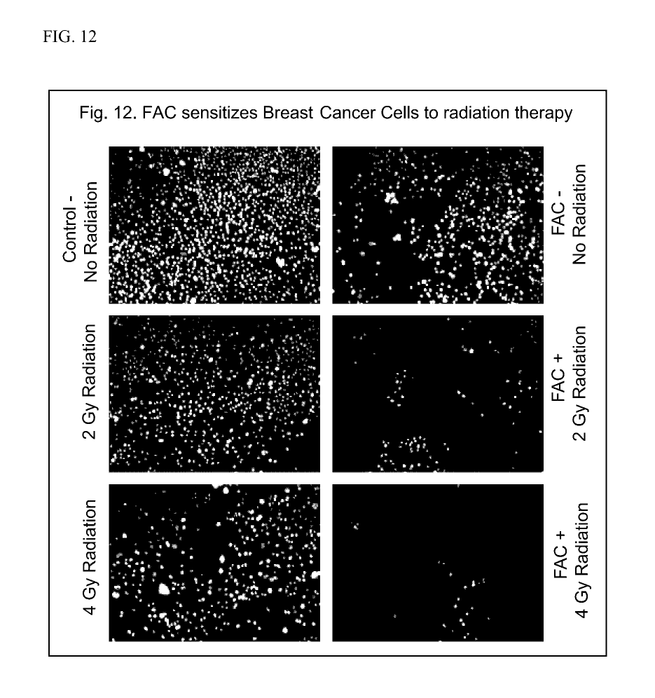

500,000 highly metastatic human breast cancer cells (MDA-MB-231-luc, Caliper Life Sciences, PerkinElmer Inc., Hopkinton, Mass.) were seeded into six-well plates in RPMI-1640 medium (3.0 ml) supplemented with 10% fetal bovine serum and antibiotics. After four hours to allow cell attachment, sets of cultures were treated with 100 μM FAC. Equal volume of complete medium was added to control cultures. One of the plates was kept aside without any radiation. Other sets of cultures were exposed to ionizing radiation (X-ray) of varying doses. After radiation, cells were cultures for five days. Live cells were stained with Hoechest 33342 dye and photographed under UV light. Representative images are shown in FIG. 12. FAC treatment potentiated the effects of radiation induced cell death. Both 2Gy and 4Gy radiation doses in the presence of FAC almost completely killed all the breast cancer cells. In contrast, significant amounts of cancer cells remained viable after radiation treatment alone. These results suggest that cell-permeable iron can be used to sensitize tumor cells to radiation therapy and improve therapeutic outcome.

### Statistics

Results represent an average of at least three independent experiments. Data are expressed as means ±SD. Differences in mean values between the two groups were analyzed using the two-tailed Student's t-test. P<0.05 was considered statistically significant.

### Example 9

Athymic mice, female (Jackson Laboratories, Bar Harbor, Me.), were transplanted with human ovarian cancer cells (MA148, Dr. S. Ramakrishnan, University of Minnesota-Twin Cities, Minneapolis, Minn.). After two weeks to allow tumor establishment, mice were divided into four groups. Group 1 was treated with saline solution and served as a control. Group 2 mice were treated with 6.8 mg/kg/day FAC intraperitoneally for three weeks. Group 3 mice were treated with carboplatin (6.8 mg/kg, twice a week for three weeks). Group 4 mice were treated with a combination of FAC and carboplatin at similar dosages. These studies demonstrated that FAC treatment improved the anti-tumor effects of carboplatin in vivo. Carboplatin is widely used to treat a number of cancer types including ovarian cancer.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method for treating a subject having a tumor, the method comprising:
co-administering to the subject cell-permeable iron and a chemotherapeutic agent or radiation treatment, each in an amount that, in combination with the other, is effective to ameliorate at least one symptom or clinical sign of the tumor.

2. The method of claim 1 wherein the cell-permeable iron and the chemotherapeutic agent or radiation are components of separate pharmaceutical compositions.

3. The method of claim 1 wherein the cell-permeable iron and the chemotherapeutic agent are administered at different sites.

4. The method of claim 1 wherein the cell-permeable iron and the chemotherapeutic agent or radiation are administered at different times.

5. The method of claim 1 wherein the cell-permeable iron and the chemotherapeutic agent and radiation are administered simultaneously.

6. The method of claim 1 wherein the cell-permeable iron and the chemotherapeutic agent are components of a single pharmaceutical composition.

7. The method of claim 1 wherein the cell-permeable iron and the chemotherapeutic agent are co-administered in amounts effective to produce a synergistic effect.

8. The method of claim 1 wherein the tumor comprises a lymphoma.

9. A method of inhibiting angiogenesis in a subject, the method comprising administering to the subject a composition comprising cell-permeable iron in an amount effective to inhibit angiogenesis.

10. The method of claim 8 wherein the method further comprises co-administering an anti-angiogenic agent.
